(19) United States
(12) Patent Application Publication
(10) Pub. No.: US 2025/0090798 A1
(43) Pub. Date: Mar. 20, 2025

(54) IMPROVED CEREBRAL ANGIOGRAPHY CATHETER

(71) Applicant: RUTGERS, THE STATE UNIVERSITY OF NEW JERSEY, New Brunswick, NJ (US)

(72) Inventor: Amit SINGLA, New Brunswick, NJ (US)

(73) Assignee: RUTGERS, THE STATE UNIVERSITY OF NEW JERSEY, New Brunswick, NJ (US)

(21) Appl. No.: 18/700,140

(22) PCT Filed: Oct. 11, 2022
(86) PCT No.: PCT/US2022/046280

§ 371 Date: Apr. 10, 2024

Related U.S. Application Data
(60) Provisional application No. 63/254,408, filed on Oct. 11, 2021.

(51) Int. Cl. A61M 25/00; A61M 25/09; A61M 25/10

(52) U.S. Cl. CPC A61M 25/0041; A61M 25/09; A61M 25/10; A61M 2025/1052

(57) ABSTRACT

Described herein is a diagnostic catheter and a catheter system including the same. The diagnostic catheter of the disclosure is configured for advancing from a first blood vessel (such as a subclavian artery or descending aorta artery) into a second blood vessel (such as a patient head or neck artery) that forms a large angle with the first blood vessel at the bifurcation point of the two blood vessels. The diagnostic catheter of the disclosure includes an inflatable balloon coupled to or formed on an exterior surface of the distal region, which is able to anchor the distal portion of the diagnostic catheter near or just inside the second blood vessel, thereby simplifying the subsequent navigation steps. Also described herein are methods of using the diagnostic catheter and the catheter system to navigate in the blood vessel networks of patients.

### CROSS-REFERENCE TO RELATED APPLICATION

The present application claims priority under 35 U.S.C. § 119(e) to U.S. Provisional Patent Application No. 63/254,408, filed Oct. 11, 2021, which is incorporated herein by reference in its entirety.

### BACKGROUND

Diagnostic catheters with a characteristic shape, such as the Simmons, vertebral or Berenstein catheters, are commonly used diagnostic catheters to gain access to the carotid arteries or vertebral arteries in the neck. These catheters are used to perform diagnostic angiograms and evaluate the anatomy and the pathology of the neck and brain blood vessels. A guide catheter, which defines a larger lumen, is typically advanced over the Simmons or other diagnostic catheter to perform brain interventions and provide treatment for brain aneurysms, arteriovenous malformations, and mechanical thrombectomy for strokes. The guide catheter, however, can be advanced only if the diagnostic catheter or the guidewire has been advanced distally into the carotid artery or subclavian artery branch entrance, so that the guide catheters have support to ride over either the guidewire, the diagnostic catheter, or both.

Advancing the diagnostic catheters into the carotid, vertebral arteries, peripheral arteries or renal arteries, however, is sometimes difficult to achieve, with setbacks being frequently encountered during this process. This can lead to loss of the precious time when the neurointerventionalist is performing typically time-critical procedures, such as stroke interventions, where the time necessary to achieve recanalization of the brain vessels is paramount. Likewise, when the interventional radiologist or a cardiologist is performing procedures such as accessing the renal arteries, the frequently encountered setbacks can make the processes significantly more difficult.

There is thus a need in the art for improved diagnostic/access catheters that are specially configured for neurointerventions/peripheral or cardiac interventions. The present disclosure addresses this need.

### BRIEF DESCRIPTION OF THE DRAWINGS

For a fuller understanding of the nature and desired objects of the present disclosure, reference is made to the following detailed description taken in conjunction with the accompanying drawing figures wherein like reference characters denote corresponding parts throughout the several views.

FIG. 1 depicts a cross-sectional perspective view of a cerebral angiography catheter system within a patient's aortic system.

FIG. 2 depicts a cross-sectional perspective view of an improved cerebral angiography catheter system within a patient's aortic system in accordance with some embodiments.

FIG. 3 depicts a diagnostic Simmons catheter.

FIG. 4 depicts distal curvatures of diagnostic catheters according to some embodiments.

FIGS. 5A-5C depict an improved diagnostic catheter according to some embodiments.

FIGS. 6A-6B demonstrate the stability conferred by the inflatable balloon to a non-limiting example of the improved diagnostic catheter in a test environment, in accordance with some embodiments.

### SUMMARY

In some aspects, the present disclosure provides a catheter.

In some embodiments, the catheter includes a catheter shaft having at least one curve near a distal end thereof.

In some embodiments, the catheter further includes an inflatable balloon coupled to or formed on an exterior surface of a distal region of the catheter shaft.

In some embodiments, an outer diameter of the catheter shaft ranges from about 1.2 mm to about 2.5 mm.

In some embodiments, an inner diameter of a catheter lumen inside the catheter shaft ranges from about 0.1 mm to about 2 mm.

In some embodiments, the curve includes a distal curve, and the inflatable balloon is between the distal curve and a distal end of the catheter shaft.

In some embodiments, the curve includes a distal curve, and the inflatable balloon is between the distal curve and a proximal end of the catheter shaft.

In some embodiments, the at least one curve includes a distal curve and a proximal curve, and the inflatable balloon is between the distal curve and the proximal curve.

In some embodiments, a curvature profile of the catheter shaft near the distal end thereof is a curvature profile of a Simmons catheter, a Berenstein catheter, an angle catheter, a tapered angle catheter, a Vitek catheter, a renal double curve catheter, a Mikaelsson catheter, and/or a Cobra catheter.

In some embodiments, a distal end of the inflatable balloon is about 100 mm or less from the distal end of the diagnostic catheter.

In some embodiments, the distal end of the inflatable balloon is about 2 mm or more from the distal end of the diagnostic catheter.

In some embodiments, a length of the balloon along a direction of the catheter shaft ranges from about 5 mm to about 20 mm.

In some embodiments, the catheter further includes an inflation lumen having an inflation opening inside the balloon and an inflation port at a proximal end of the catheter shaft, wherein the inflation lumen and the inflation port inflates or deflates the balloon by passing an inflation fluid across the inflation opening.

In some embodiments, the catheter further includes a radiopaque marker at or near the distal end of the catheter shaft.

In some embodiments, the catheter is a diagnostic angiography catheter.

In some aspects, the present disclosure provides a catheter system. In some embodiments, the catheter system includes a catheter, such as the catheter described herein.

In some embodiments, the catheter includes a catheter shaft having at least one curve near a distal end thereof.

In some embodiments, the catheter further includes an inflatable balloon coupled to or formed on an exterior surface of a distal region of the catheter shaft.

In some embodiments, the catheter system further includes a guide wire moveable alone a longitudinal direction inside a lumen of the catheter shaft.

In some embodiments, a diameter of the guide wire ranges from about 0.3 mm to about 1 mm.

In some embodiments, the catheter system further includes a guide catheter having a guide lumen, wherein the catheter and/or the guide wire is movable alone a longitudinal direction inside the guide lumen.

In some embodiments, a length of the guide catheter ranges from about 500 mm to about 1100 mm.

In some embodiments, an outer diameter of the guide catheter ranges from about 1.8 mm to about 3 mm.

In some embodiments, the inner diameter of the guide catheter ranges from about 1.6 mm to about 2.4 mm.

In some aspects, the present disclosure provides a method of navigating the catheter system from a first blood vessel across a bifurcation point to a second blood vessel.

In some embodiments, method includes the steps of: advancing the guide wire from the first blood vessel toward the second blood until the distal end of the guide wire passes the bifurcation point; advancing the diagnostic catheter along the guide wire until the inflatable balloon reaches the bifurcation point or the second blood vessel; inflating the inflatable balloon such that the inflatable balloon anchors the diagnostic catheter to an inner wall of the bifurcation point or the second blood vessel; advancing the guidewire inside the diagnostic catheter further into the second blood vessel; deflating the balloon to release the diagnostic catheter from the inner wall of the bifurcation point or the second blood vessel; and advancing the diagnostic catheter further into the second blood vessel using the guidewire as a guide.

In some embodiments, the method further includes: advancing the diagnostic catheter from the second blood vessel into a third blood vessel, or advancing the diagnostic catheter from the third blood vessel into a fourth blood vessel following the same steps for advancing the diagnostic catheter from the first blood vessel in to the second blood vessel.

In some embodiments, an angle between the first blood vessel and the second blood vessel, an angle between the second blood vessel and the third blood vessel, and/or an angle between the third blood vessel and the fourth blood vessel are about 45 degrees or higher.

In some embodiments, the first blood vessel is the aorta, and the second blood vessel is a common carotid artery, an innominate artery, a subclavian artery, a renal artery, or another peripheral artery connected to the aorta.

In some embodiments, the third blood vessel is an internal carotid artery, external carotid artery, subclavian artery, or vertebral artery.

In some embodiments, the fourth blood vessel is a vertebral artery.

In some embodiments, the method further includes advancing a guide catheter over the diagnostic catheter and/or the guide wire.

### Definitions

The instant disclosure is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

The study described herein (“the present study”), in one aspect, addressed one of the sources of difficulty for advancing diagnostic catheters from a first blood vessel into the carotid or vertebral arteries, or peripheral or visceral arteries such as a renal artery.

When navigating a diagnostic catheter (such as a diagnostic Simmons catheter) in a blood vessel, a guidewire is typically advanced through the catheter first. Once the guidewire has obtained the distal purchase, the catheter can be advanced distally toward the destination over the guidewire.

Normally, the diagnostic catheters, such as diagnostic double curve catheters, can be advanced into the proximal portion or entrance of the carotid artery, the vertebral arteries, the subclavian arteries, or other peripheral arteries (such as renal arteries) with the help of the characteristic shapes of the catheters.

At this point, however, due to the tortuous nature of the blood vessels networks ahead, advancing the catheter further into the carotid or vertebral arteries can become difficult. After the guidewire is advanced into the carotid artery, vertebral arteries, subclavian arteries, or other peripheral arteries and after the tip of the diagnostic catheter advancing over the guidewire just passes the bifurcation point, the diagnostic catheter is still in an unstable position. Moreover, the sharp turns at the bifurcation point mean that (a) the direction of the force applied to advance the guidewire and/or diagnostic catheter, and (b) the direction towards which the tip of the diagnostic catheter needs to move, differ significantly, sometimes by more than 90 degrees.

For at least the above reasons, at this point attempts to further advance the guidewire, or advance the diagnostic catheter along the guidewire, often cause the tip of the diagnostic catheter that just entered the carotid, vertebral, subclavian or renal arteries to fall back into the blood vessel from where these blood vessels originate, such as aorta. Furthermore, the diagnostic catheter that fell back into the aorta sometimes drags the guidewires back, as well. When the above happens, the navigation attempt of the diagnostic catheter suffers a catastrophic setback.

Such setbacks can make advancement of the guide catheters significantly more difficult, resulting in multiple attempts to achieve the distal access, and sometimes not allowing for the procedures to be completed at all.

The present study provides solutions to the above identified source of problem. The solutions include a novel diagnostic catheter, the distal end of which can be securely anchored in the target blood vessel (such as the carotid, innominate, subclavian, renal arteries or other types of peripheral arteries) at locations just past the bifurcation point from the blood vessel of entry. This allows for guidewires to further advance without pulling the diagnostic catheter back. The present study further provides methods of using such diagnostic catheters.

The diagnostic catheters and methods herein are not limited to any specific blood vessels. As detailed herein, the present disclosure is useful in any situation where a diagnostic catheter needs to be navigated from a first blood vessel into a second blood vessel that forms an angle (such as, but not limited to, equal to or greater than about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160. 165, 170, 175, or 180 degrees) with the first blood vessel by crossing a bifurcation point connecting the two blood vessels. As described herein, various features of the diagnostic catheter herein allow the navigation into the second blood vessel (and the subsequent navigation into the third or fourth blood vessel) to become substantially easier.

### Diagnostic Catheter

In some aspects, the present disclosure is directed to an improved diagnostic catheter. In some embodiments, the diagnostic catheter herein can be used (with or without the aid of additional articles such as, but not limited to, a guide wire and/or a guide catheter) to navigate from a first blood vessel into a second blood vessel (or from a second to a third blood vessel, or from a third to a fourth blood vessel) forming an angle with the first, second, or third blood vessel and across a bifurcation point connecting the two blood vessels.

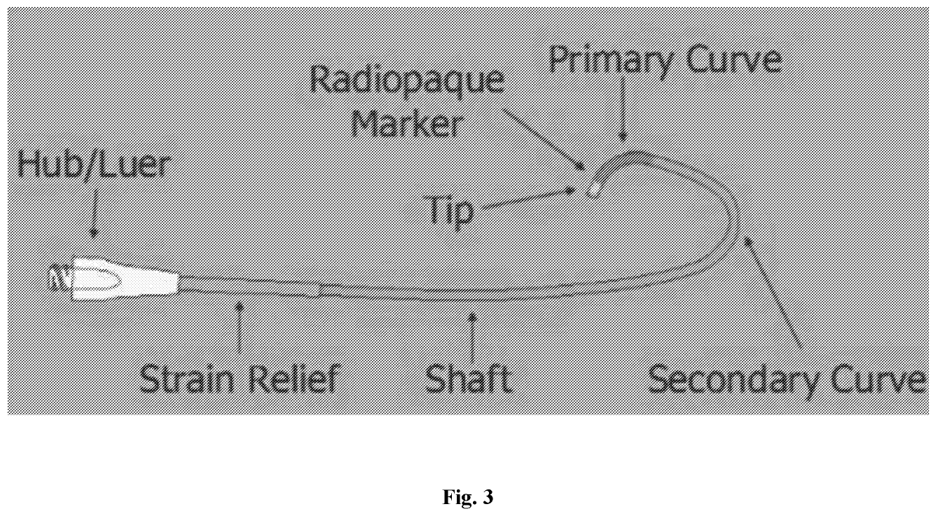

In some embodiments, the improved diagnostic catheter has advantages over conventional diagnostic catheters such as Simmons catheters (e.g., where a Simmons catheter has a distal curve and a proximal curve with a straight segment in between those curves), Berenstein catheters, renal double curve catheters, or other similar catheters with curves at the distal portions thereof for ease of navigation in blood vessel systems involving bifurcation points and sharp turns. An exemplary conventional diagnostic catheter is shown in FIG. 3.

As used herein, the term “distal end”, when used to describe catheters and/or guidewires, refers to the end configured to be inserted into the body of a patient, and the term “distal” refers to proximity to the distal end.

The term “proximal end,” when used to describe catheters and/or guidewires, refers to the end configured to be left outside the patient's body, often configured to be directly operated by the physician, and the term “proximal” refers to proximity to the proximal end.

In some embodiments, the improved characteristics of the present catheter is suitable for use in endovascular medical procedures. In some embodiments, the improved diagnostic catheter herein is a cerebral angiography catheter system.

In some embodiments, the improved diagnostic catheter includes an inflatable balloon near the distal end thereof In some embodiments, the improved diagnostic catheter includes the distal curve and a proximal curve featured in, for example, Simmons catheters. In some embodiments, the inflatable balloon is located along the straight segment of the diagnostic catheter between the proximal curve and the distal curve.

In some embodiments, the improved diagnostic catheter is configured such that the balloon can be inflated once the proximal cervical carotid, vertebral or subclavian vessel is catheterized. The inflated balloon can anchor the catheter to the walls of the vessels from inside, thereby stabilizing the diagnostic catheter. While the diagnostic catheter is stabilized in the vessels with the balloon inflated, a guidewire can be advanced distally inside the diagnostic catheter. Once the guidewire has been advanced into the distal neck blood vessels/vessel of interest/branch vessel and the purchase has been obtained, the balloon can then be deflated. Due to the anchoring provided by the balloon, the guidewire can advance with relative ease without causing the diagnostic catheter to fall back.

When the guidewire has advanced sufficiently far so that the guidewire can provide enough support for the movement of diagnostic catheter across the bifurcation point, the diagnostic catheter (after deflating the balloon) can be advanced over the guide wire distally to reach the desired destination and allow the diagnostic angiograms to be performed. This can also help in advancing the guide catheters once the diagnostic catheter has been advanced distally to perform the procedures and interventions to deal with strokes and other brain vascular malformations or peripheral or cardiac interventions.

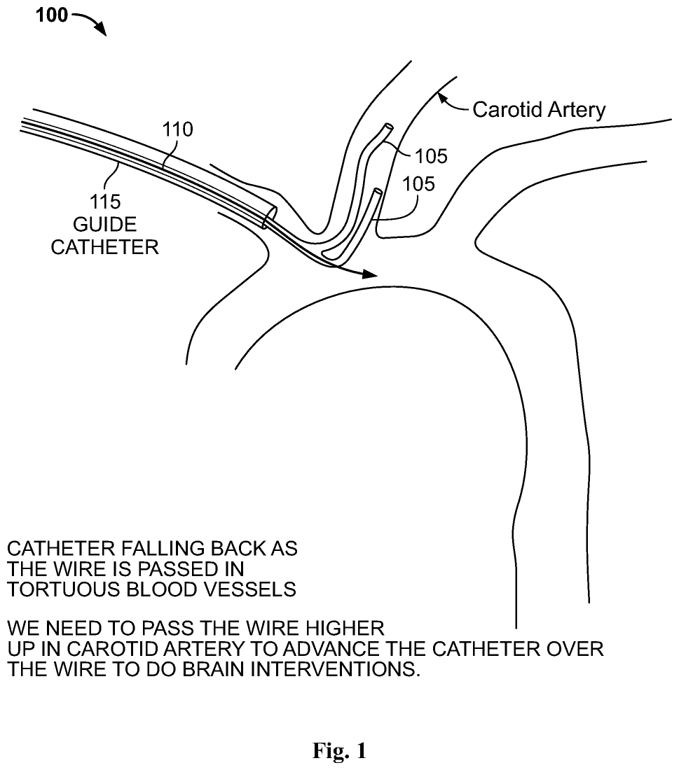

FIG. 1 depicts a cross-sectional perspective view of a cerebral angiography catheter system within a patient aortic system including a diagnostic catheter. As shown in FIG. 1, the catheter system 100 including a diagnostic catheter 105, a guide wire 110, and optionally a guide catheter 115, is passed through a branch of the aortic system, such as through a subclavian artery (e.g., with access via a patient's arm), and navigated into the carotid artery. As can be seen in FIG. 1, access to a carotid artery from the subclavian artery requires an angled pathway for the catheter system.

Typically, a catheter system is positioned in or translated through an artery in a piecewise fashion. The diagnostic catheter 105 defines a diagnostic lumen, which the guide wire 110 is passed through. The guide wire 110 provides the diagnostic catheter 105 with rigidity as the catheter 105 translates through the artery, and the diagnostic catheter 105 provides an intended angle or direction for which the guide wire 110 can exit the distal end of the diagnostic catheter 105. The diagnostic catheter 105 is typically passed into the carotid artery first, due to its shape and flexibility. Then, the guide wire 110 is passed through the diagnostic catheter 105, around the subclavian/carotid artery convergence, and into the carotid artery. The distal end of the guide wire 110 then passes external to the distal end of the diagnostic catheter, further into the artery, which assists in guiding the diagnostic catheter 105 further into the artery.

However, based on the sharp angle created by the subclavian/carotid artery convergence, especially in the tortuous vessels, the distal end of the diagnostic catheter 105 can “fall out” of the carotid artery when the guide wire 110 is passed through the lumen of the diagnostic catheter 105. In particular, when the distal end of the guide wire 110 reaches the subclavian/carotid artery convergence, the rigidity of the guide wire 110 can potentially push the distal end of the more flexible diagnostic catheter 105 out of the carotid artery. This can result in multiple attempts to successfully pass the guide wire 110 into the carotid artery. As the angiography procedure is typically a time-critical event, multiple attempts to successfully position the diagnostic catheter 105 can result in decreasingly positive outcomes for a patient.

Guidewires typically used for neurointerventions along with the diagnostic/access catheters can be 0.035 inches in diameter and 150 cm long. They can have a super-elastic nitinol core with hydrophilic coating to provide a lubricious, low-friction feel inside the vessel. The angled tip of the guidewires facilitates access into the vessels as they branch off from the parent vessel. The length of the flexible distal tip can vary from 3-8 cm to help navigate the tortuous vessels coming off from the aortic arch. However, even with these features, they have limitations and without the support from the stable catheter, often times they cannot be advanced distally to gain purchase to help advance the catheter.

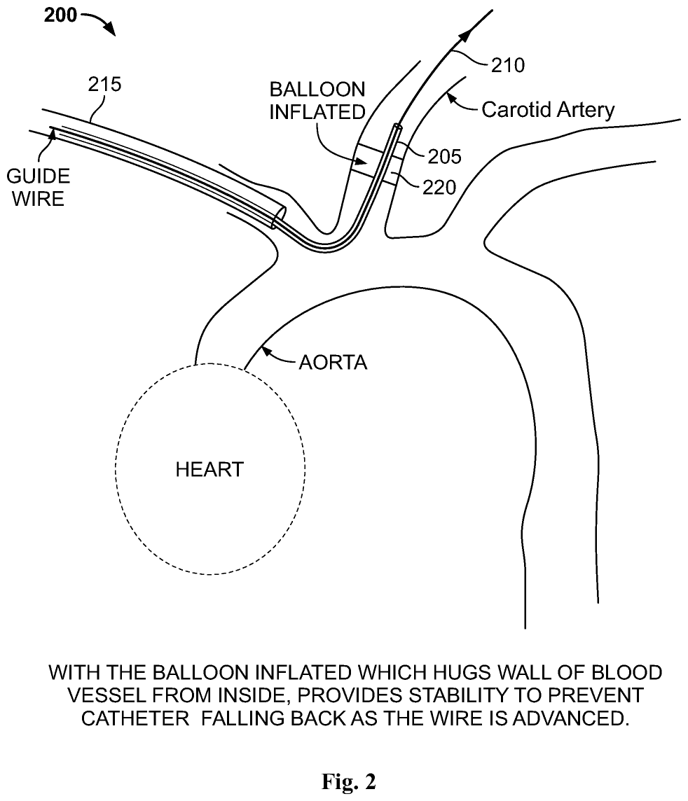

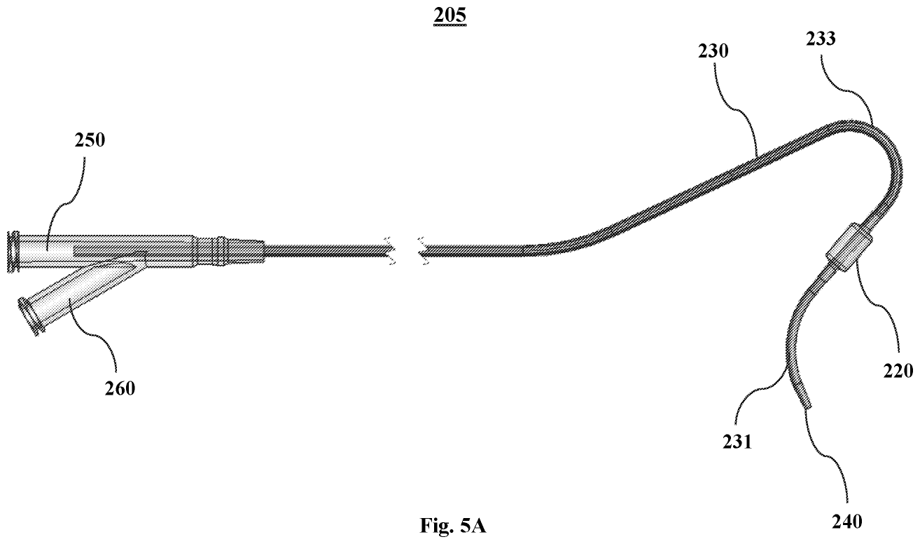

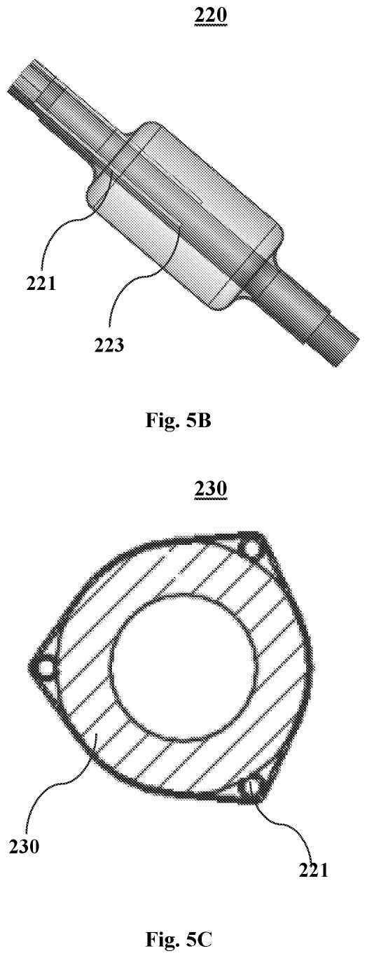

FIG. 2 depicts a cross-sectional perspective view of a catheter system 200 including a non-limiting example of the improved diagnostic catheter herein within a patient aortic system according to an embodiment of the present disclosure. FIG. 5 depicts a non-limiting example of the improved diagnostic catheter outside the body of a patient.

In some embodiments, the catheter system includes the improved diagnostic catheter 205, a guide wire 210, and a guide catheter 215. In some embodiments, the catheter 205 includes an expandable balloon 220 coupled to a portion of the exterior surface of the diagnostic catheter 205. The aggregated cross-sectional diameter of the diagnostic catheter 205 and the inflatable balloon 220, when deflated, can be less than the cross-sectional diameter of the lumen defined by the carotid artery and the subclavian artery. Further, expanding the balloon 220 can increase the aggregated cross-sectional diameter to be equal to, or greater than, the cross-sectional diameter of the carotid artery lumen (e.g., when the carotid artery lumen is in an “unexpanded” state from the balloon).

Catheter Shaft In some embodiments, the diagnostic catheter 205 includes a catheter shaft enclosing a catheter lumen.

In some embodiments, a length of the catheter shaft ranges from about 800 mm to about 1500 mm, such as from about 850 mm to about 1400 mm, or from about 900 mm to about 1300 mm.

In some embodiments, an outer diameter of the shaft ranges from about 1.2 mm to about 2.5 mm, such as: from about 1.3 mm to about 2.3 mm; from about 1.4 mm to about 2.1 mm; or from about 1.5 mm to about 2.0 mm.

In some embodiments, an inner diameter of the catheter lumen ranges from about 1 mm to about 2 mm, such as: about 0.6 mm to about 1.4 mm; about 0.7 mm to about 1.3 mm; or about 0.8 mm to about 1.2 mm.

In some embodiments, the catheter shaft comprises a hydrophilic coating on an outer surface thereof along the length of the catheter shaft. In some embodiments, the hydrophilic coating reaches the distal end of the catheter shaft. In some embodiments, a length of the hydrophilic coating along the longitudinal direction ranges from about 80 mm to about 300 mm, such as from about 90 mm to about 275 mm, or from 100 mm to about 250 mm, such as from the distal end of the catheter shaft.

Curves and Bends Near Distal End In some embodiments, the diagnostic catheter 205 includes one or more curves or bends near the distal end thereof, so as to reduce the difficulties of passing through a bifurcation point and enter a second (or third or fourth) blood vessel in a direction significantly different from a first (or second or third) blood vessel where the catheter 205 is currently located. In some embodiments, an angle between the first blood vessel and the second blood vessel (or between the second blood vessel and third blood vessel, or between the third and fourth blood vessel) is about 45 degrees or higher, such as about 60 degrees or higher, about 90 degrees or higher, or about 120 degrees or higher.

In some embodiments, the diagnostic catheter 205 is configured for translation through the head and neck arteries of the aortic system, such as having curves or bends that facilitate the passage of the catheter 205 through a particular head or neck artery. In some embodiments, the curves and bends of the diagnostic catheter 205 are configured for neurointerventions. In some embodiments, the curves and bends of the diagnostic catheter 205 are configured for peripheral endovascular procedures.

In some embodiments, a distance between the curves and/or bends of the diagnostic catheter 205 and the distal end of the diagnostic end of the catheter 205 are from about 5 mm to about 100 mm (such as from about 10 mm to about 80 mm or from about 20 mm to about 60 mm).

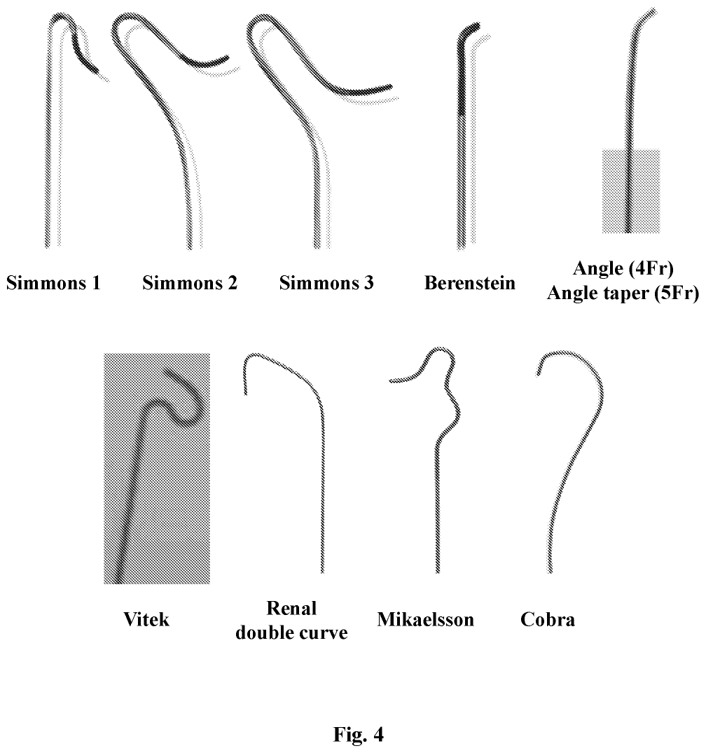

In some embodiments, the configuration of the distal end of the diagnostic catheter 205 is based on the configuration of a Simmons catheter, which can include a shape intended for translation through the carotid artery. In some embodiments, the catheter 205 is the same as a Simmons catheter except for the added inflatable balloon near the distal end thereof In some embodiments, according to the configuration of Simmons catheters, the catheter 205 can include one or more curve or bends along the catheter length. In some embodiments, according to the configuration the Simmons catheter, the catheter 205 can include a proximal curve and a distal curve. In some embodiments, according to the configuration of Simmons catheters, the body of the catheter 205 is substantially straight, and the proximal curve can curve the catheter 205 into the shape of a hook, such that the distal region of the catheter loops back towards the body. In some embodiments, the distal curve can curve the distal end of the catheter outwardly away from the catheter body. The Simmons catheter the configuration of the catheter 205 is based on, for example, Simmons 1, 2, and 3. For example, the catheter can include a proximal curve and a distal curve, with a straight segment between the curves. The curvature of the Simmons catheters at the distal end is shown in FIG. 4. Simmons catheters are described in, for example, Simmons et al. (Am JRoentgenol Radium Ther Nucl Med. 1973 November; 119(3):605-12. doi: 10.2214/ajr.119.3.605), the entirety of said reference is hereby incorporated herein by reference.

In some embodiments, the configuration of the distal end of the diagnostic catheter 205 is based on the configuration of the Berenstein catheter. The Berenstein catheter can include a proximal curve in the distal region of the catheter that curves the distal end away from the catheter body length. The curvature of the Berenstein catheter at the distal end is shown in FIG. 4. In some embodiments, the curved configurations described in this paragraph and the previous paragraph are suitable for facilitating the passage through a particular head or neck artery, among others.

In some embodiments, the configurations of the distal end of the diagnostic catheter 205 are based on the configurations of the angle catheter, the angle taper catheter, and/or the Vitek diagnostic catheter. Angle catheters have a curve in the distal region of the catheter, similar to the Berenstein catheter. The Vitek diagnostic catheter has a complex U-shaped curve in the distal region. All these diagnostic catheters typically used for neurointerventions are 5 french (5F) in luminal diameter (The French scale, French gauge or Charrière system measures the outer diameter of a catheter, and is defined as 1 F=⅓ mm), however they can be 4 F or 6 F as well. The curvature of the angle catheter, the angle taper catheter and/or the Vitek diagnostic catheter at the distal ends thereof are shown in FIG. 4. In some embodiments, the curved configurations described in this paragraph are suitable for neurointerventions, among others.

In some embodiments, the configuration of the distal end of the diagnostic catheter 205 is based on another double curve diagnostic catheters, such as a renal double curve catheter, a Mikaelsson catheter, or a Cobra catheter. The curvature of the renal double curve catheter, the Mikaelsson catheter, and/or the Cobra catheter at the distal ends thereof are shown in FIG. 4. In some embodiments, the curved configurations described in this paragraph are suitable for peripheral endovascular procedures, among others.

It is worth noting that, without the novel features of the present improved diagnostic catheter, each of the conventional catheters described herein can be difficult to use in complex aortic arch anatomy, resulting in multiple attempts or sometime failure to achieve the goal of performing mechanical thrombectomy for stroke, aneurysm, or other brain vascular malformations embolizations, or performing diagnostic angiograms. The distal portion curvature characteristics of these conventional catheters, however, can be included in the present improved diagnostic catheter to further simply the navigation into the second blood vessel.

Inflatable Balloon Referring again to FIG. 2, in some embodiments, the diagnostic catheter 205 includes inflatable balloon 220 in a distal region of the diagnostic catheter 205.

In some embodiments, the inflatable balloon 220 is in a distal region of the diagnostic catheter 205.

In some embodiments, the distal end of the inflatable balloon 220 is about 100 mm or less (such as: about 90 mm or less; about 80 mm or less; about 70 mm or less; about 60 mm or less; about 50 mm or less; about 40 mm or less; about 30 mm or less; about 20 mm or less; about 10 mm or less; or about 5 mm or less) from the distal end of the diagnostic catheter 205.

In some embodiments, the distal end of the inflatable balloon 220 is about 2 mm or more (such as: about 5 mm or more; about 10 mm or more; about 20 mm or more; or about 30 mm or more) from the distal end of the diagnostic catheter 205.

In some embodiments, the length of the balloon 220 along a direction of the catheter 205 ranges from about 5 mm to about 20 mm, such as: from about 6 mm to about 18 mm; from about 7 mm to about 15 mm; or from about 8 mm to about 12 mm.

As detailed elsewhere herein, in some embodiments, the diagnostic catheter 205 includes one or more curves near the distal end thereof. Accordingly, in some embodiments, the balloon 220 is proximal to the distal-most curve. In some embodiments, the balloon 220 distal to the distal-most curve.

In some embodiments, the diagnostic catheter includes two curves, such as in the case of Simmons catheter-style curves. In some embodiments, the inflatable balloon 220 is positioned between a proximal curve and a distal curve of the catheter distal region.

Although inflatable balloons are not known to be used in diagnostic catheters, such structures are used in non-diagnostic balloon catheters. As such, in some embodiments, the inflatable balloon 220 includes characteristics of non-diagnostic balloon catheters, such as those described elsewhere herein.

In some embodiments, the balloon is a compliant balloon or a non-compliant balloon. In some embodiments, a material of the balloon comprises polyurethane, silicone, or another compliant (and/or non-compliant) material.

Balloon catheters come as guide catheters (BGC) or microcatheters. Balloon guide catheters (e.g., Cello™) can range in size from 6 F to 9 F with an outer diameter ranging from 0.079 to 0.114 inch. The length of the compliant silicone balloon can range from 7-10 mm. A Walrus BCG can include a polyurethane compliant balloon which can inflate to a diameter of 11.1 mm. The length of such BGC is typically in the range of 90-95 cm. They have a straight tip and they are typically advanced over the diagnostic/access catheters. Because they are stiffer (e.g., to provide support), they by themselves are unable to be advanced into the distal neck vessels without the support of the diagnostic/access catheters and the guidewires.

Other balloon catheters for neurointerventions can include microcatheters such as SCEPTER™ (Microvention) which harbors a balloon that can range from 4-4.5 mm in diameter and length from 10-20 mm. The balloons can inflate ranging from 2-6 mm with a nominal diameter of 4-4.5 mm. Other examples of balloon microcatheters used for neurointerventions can include HYPERFORM™ (Medtronic) and TRANSFORM™ (Stryker). These balloon catheters also have a balloon that can range from 3-7 mm in diameter while inflated. These balloon catheters are used to provide support to the embolization material inside the small blood vessels in the brain while the vascular malformations (such as aneurysms and arteriovenous malformations) are embolized. Some of these balloon microcatheters have a single lumen (e.g., HYPERFORM™ and TRANSFORM™) and some have a dual lumen (e.g., SCEPTER™). Dual lumen microcatheters can include a separate inflation port at the proximal hub of the balloon catheter along with a lumen for a micro-guidewire. Single lumen balloon microcatheters can include one lumen and a single port at the proximal hub used for advancing the microwire as well as inflating the balloon. The balloon in single lumen microcatheters can inflate while having the guidewire inside the lumen of the microcatheter.

### Additional Structures

Referring now to FIG. 5A, a catheter 205 is illustrated. In some embodiments, the catheter 205 further includes a radiopaque marker 240. In some embodiments, the radiopaque marker 240 is opaque to X-rays or other types of radiations. For example, when radiopaque marker 240 is opaque, the visibility of the catheter 205 can be increased during radiographic imaging when the catheter 205 or portions thereof is inside the body of a patient. In some embodiments, the radiopaque marker 240 is located at the distal-most portion of the diagnostic catheter 205.

In some embodiments, the catheter 205 further includes a guide access port 250. In some embodiments, the guide access port 250 is configured to allow a guide wire (e.g., guide wire 210 illustrated in FIG. 2) to move inside the lumen of the shaft 230.

Referring to FIGS. 5A-5C, in some embodiments, the catheter 205 further includes an inflation lumen 221 having an inflation opening 223 inside the balloon 220. In some embodiments, the catheter 205 further includes an inflation port 260. In some embodiments, an inflation fluid injected into the inflation portion 260 passes through the inflation lumen 221 and enters the balloon 220 via the opening 223. In some embodiments, the inflation fluid is water, a saline solution, a gas (e.g., an inert gas, a reactive gas, etc.), or air.

Referring now to FIG. 5C, in some embodiments, the inflation lumen 221 runs inside a side wall of the shaft 230. However, the configuration of the inflation lumen 221 is not limited thereto. One of ordinary skill in the art would understand that the inflation lumen 221 can run outside the shaft 230, or inside the lumen of the shaft 230.

In some embodiments, the guide wire 210 (e.g., see FIG. 2) is not considered a part of the diagnostic catheter 205. In some embodiments, the diagnostic catheter 205 includes the guide wire 210.

In some embodiments, the diameter of the guide wire 210 ranges from about 0.3 mm to about 1 mm, such as from about 0.4 mm to about 0.9 mm, or from about 0.5 mm to about 0.8 mm.

In some embodiments, the guide catheter 215 (e.g., see FIG. 2) is not considered a part of the diagnostic catheter 205. In some embodiments, the diagnostic catheter 205 includes the guide catheter 215.

In some embodiments, the length of the guide catheter 215 ranges from about 500 mm to about 1100 mm, such as from about 600 mm to about 1000 mm, or from about 700 mm to about 900 mm.

In some embodiments, the outer diameter of the guide catheter 215 ranges from about 1.8 mm to about 3 mm, such as from about 1.8 mm to about 2.8 mm, from about 1.9 mm to about 2.6 mm, or from about 1.8 mm to about 2.4 mm.

In some embodiments, the inner diameter of the guide catheter 215 ranges from about 1.6 mm to about 2.4 mm, such as about 1.6 mm to about 2.2 mm, about 1.7 mm to about 2.0 mm, or about 1.6 mm to about 1.9 mm.

Improved Catheter System In some aspects, the present disclosure is directed to an improved catheter system.

In some embodiments, the improved catheter system includes an improved diagnostic catheter and a guide wire.

In some embodiments, the improved catheter system further includes a guide catheter.

In some embodiments, the improved diagnostic catheter, the guide wire and/or the guide catheter are the same as or similar to those described elsewhere herein, such as in the “Improved Diagnostic Catheter” section.

### Method of Navigating Catheter

In some aspects, the present disclosure is directed to a method of navigating a diagnostic catheter in a blood vessel network with sharp turns and bifurcation points. In some embodiments, the navigation of the diagnostic catheter is aided by a guide wire (e.g., guide wire 210) and/or a guide catheter (e.g., guide catheter 215). In some embodiments, the improved diagnostic catheter, the guide wire and/or the guide catheter are the same as or similar to those described elsewhere herein, such as in the “Improved Diagnostic Catheter” and the “Improved Catheter System” sections.

In some embodiments the method is a method of navigating the diagnostic catheter from a first blood vessel into a second blood vessel across a bifurcation point where the two blood vessels connect. In some embodiments, the angle between the first blood vessel and the second blood vessel at the bifurcation point is equal to or greater than about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160. 165, 170, 175, or 180 degrees. In some embodiments, the angle between the first blood vessel and the second blood vessel at the bifurcation point is about 45 degrees or higher, such as about 60 degrees or higher, about 90 degrees or higher, or about 120 degrees or higher.

In some embodiments, the method includes advancing a guide wire (e.g., guide wire 210) along the first blood vessel until the guide wire crosses the bifurcation point and enters the second blood vessel. In some embodiments, at this stage, the distal end of the guide wire passes the bifurcation point for about 10 mm or more, such as: about 15 mm or more; about 20 mm or more; about 25 mm or more; or about 30 mm or more.

In some embodiments, the method further includes advancing the diagnostic catheter over the guide wire until the inflatable balloon of the diagnostic catheter is near the bifurcation point or in the second (or the third) blood vessel.

In some embodiments, the method further includes inflating the inflatable balloon such that the inflatable balloon anchors the diagnostic catheter to an inner wall of the second (or the third) blood vessel or the vessel structure at the bifurcation point.

In some embodiments, the method further includes advancing the guide wire further into the second (or the third) blood vessel until the guide wire is able to provide sufficient support for an advancing movement of the diagnostic catheter across the bifurcation point of the first, second and/or third blood vessel.

In some embodiments, the method further includes deflating the balloon and advancing the diagnostic catheter further into the second blood vessel over the guide wire once the guide wire is able to provided sufficient support for an advancing movement of the diagnostic catheter across the bifurcation point.

In some embodiments, a guide catheter is advanced in the first blood vessel over the diagnostic catheter and/or the guide wire. In some embodiments, the guide catheter is advanced close to the bifurcation point over the guide wire and/or the diagnostic catheter.

In some embodiments, the guide catheter is further advanced into and along the second blood vessel over the guide wire and/or the diagnostic catheter once the guide wire and/or the diagnostic catheter has provided sufficient support for an advancing movement of the guide catheter across the bifurcation point and along the length of second and/or subsequently along the third blood vessel.

In some embodiments, the first blood vessel is the aorta, the second blood vessel is a common carotid artery, and the third blood is an internal carotid or external carotid artery. In some embodiments, the first blood vessel is the aorta, the second blood vessel is the subclavian artery, and the third blood vessel is the vertebral artery. In some embodiments, the first blood vessel is the aorta, the second blood vessel is an innominate artery, the third blood vessel is the subclavian artery, and the fourth blood vessel is the vertebral artery. In some embodiments, the first blood vessel is the aorta and the second blood vessel is a renal artery. In some embodiments, the second blood vessel is another peripheral artery connected to the first blood vessel (e.g., the aorta).

FIG. 2 illustrates the navigation of a non-limiting example of improved catheter 205 from an artery of the aortic system in to a carotid artery.

Referring to FIG. 2, in some embodiments, the improved catheter 205 is inserted into a patient via an artery of the patient's aortic system. The catheter 205 can be translated through the artery lumen and towards a head/neck artery entrance. The distal region of the catheter can be positioned in the head/neck artery entrance. In some cases, the shape of the catheter distal region may aid in this positioning (e.g., the distal and proximal curves of the Simmons catheter, the curve of the Berenstein catheter, and the like). As the inflatable balloon 220 is coupled to the distal region of the catheter, the inflatable balloon 220 is also positioned in (or just beyond) the head/neck artery entrance.

Once the inflatable balloon 220 is positioned inside (or just beyond) the head/neck artery entrance, the inflatable balloon 220 can be inflated. The balloon diameter can expand to the diameter of the distal region of the catheter, to where the distal region diameter is equal to, or slightly greater than, the lumen diameter defined by the head/neck artery entrance (e.g., where the lumen diameter is the diameter is a non-expanded state). The balloon inflation can secure the distal region of the catheter in the head/neck artery entrance (or just beyond it). The guide wire 210 can then be passed through the catheter lumen and into the head/neck artery entrance. As the inflatable balloon 220 secures the distal region of the catheter to the head/neck artery, balloon inflation can prevent the catheter 205 from “falling out” of the artery entrance. The guide wire 210 can then pass through the distal end of the catheter and into the artery lumen. Once the guide wire 210 is through the catheter distal end far enough to get a purchase in the vessel and provide support to advance the diagnostic catheter, the balloon 220 can be deflated, thereby loosening (or “unsecuring”) the distal region of the catheter from the head/neck artery entrance. The diagnostic catheter 205 can then be further advanced over the guide wire 210 and through the artery to perform the angiography procedure. Once the diagnostic catheter 205 is further along the vascular tree along with the guidewire, guide catheter 215 can also be advanced distally to perform the necessary interventions.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

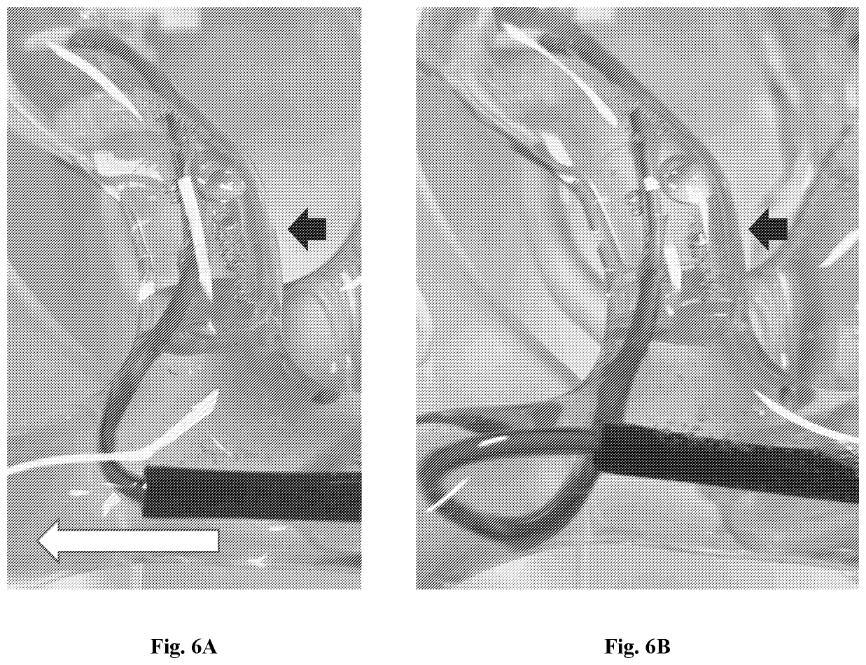

Referring to FIGS. 6A-6B, a prototype of the improved diagnostic catheter according to some embodiments was manufactured with the double curve distal profile of the Simmons catheters. The inflatable balloon near the distal end of the improved diagnostic catheter is a compliant balloon made of polyurethane. The balloon is secured to the outer surface of the improved diagnostic catheter, at the straight segment between the distal curve and the proximal curves, using heat molding. The balloon is inflated using a small tube that was attached to the outer surface of the Simmons catheter terminating in the balloon attached to the straight segment between the proximal and distal curves.

The prototype was tested in a bench-top model simulating the vasculature with large lumen vessels and branch vessels. The balloon can be inflated up to 14 mm in diameter without bursting. The balloon is 1 cm in length. The balloon was tested for stability manually. As the double-curve catheter was pushed from the proximal end (the direction of the force is indicated with a white arrow in FIG. 6A), the tip of the catheter was stable and did not herniate out of the branch vessel due to the stability provided by the inflated balloon (see the black arrows in FIGS. 6A and 6B). When the balloon was deflated and the catheter was pushed from the hub, the catheter tip herniated out of the branch vessel.

During the test, the testers were neither able to advance the catheter nor pull the catheter with the balloon inflated with ease. This benchtop experiment demonstrated that the balloon when inflated provides desirable stability to the catheter.

### Example 2

A balloon occlusion test is performed in the carotid artery to check if the patient would tolerate sacrificing the carotid artery if the patient has a large aneurysm or a tumor that is engulfing the artery. The balloon occlusion test is performed using two separate catheters including a guide catheter and a microcatheter.

With a certain balloon diagnostic catheter of the present disclosure, the same procedure can be performed with one such catheter without the need for two different catheters which can make the procedure easier and more economical.

Currently due to the curvature of the vessels (as they are originating from the arch of the aorta), it is difficult to access the left vertebral artery from the right radial artery approach which is typically used for performing cerebral angiograms. However, with a certain catheter of the present disclosure providing stability in the subclavian artery, the left vertebral artery can be accessed to perform the angiograms and brain vascular interventions.

### EQUIVALENTS

Although selected illustrative embodiments of the disclosure have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### ENUMERATED EMBODIMENTS

The following enumerated embodiments are provided, the numbering of which is not to be construed as designating levels of importance. In some aspects, the present disclosure is directed to the following non-limiting embodiments:

Embodiment 1 provides a catheter comprising:
a catheter shaft having at least one curve near a distal end thereof; and
an inflatable balloon coupled to or formed on an exterior surface of a distal region of the catheter shaft.

Embodiment 2 provides the catheter of embodiment 1, wherein an outer diameter of the catheter shaft ranges from about 1.2 mm to about 2.5 mm.

Embodiment 3 provides the catheter of any one of embodiments 1-2, wherein an inner diameter of a catheter lumen inside the catheter shaft ranges from about 0.1 mm to about 2 mm.

Embodiment 4 provides the catheter of any one of embodiments 1-3, wherein at least one of the following applies:
(a) the curve comprises a distal curve, and the inflatable balloon is between the distal curve and a distal end of the catheter shaft,
(b) the curve comprises a distal curve, and the inflatable balloon is between the distal curve and a proximal end of the catheter shaft,
(c) the at least one curve comprises a distal curve and a proximal curve, and the inflatable balloon is between the distal curve and the proximal curve.

Embodiment 5 provides the catheter of any one of embodiments 1-4, wherein a curvature profile of the catheter shaft near the distal end thereof is a curvature profile of a Simmons catheter, a Berenstein catheter, an angle catheter, a tapered angle catheter, a Vitek catheter, a renal double curve catheter, a Mikaelsson catheter, and/or a Cobra catheter.

Embodiment 6 provides the catheter of any one of embodiments 1-5, wherein a distal end of the inflatable balloon is about 100 mm or less from the distal end of the diagnostic catheter, and wherein the distal end of the inflatable balloon is about 2 mm or more from the distal end of the diagnostic catheter.

Embodiment 7 provides the catheter of any one of embodiments 1-6, wherein a length of the balloon along a direction of the catheter shaft ranges from about 5 mm to about 20 mm.

Embodiment 8 provides the catheter of any one of embodiments 1-7, which further comprises an inflation lumen having an inflation opening inside the balloon and an inflation port at a proximal end of the catheter shaft, wherein the inflation lumen and the inflation port inflates or deflates the balloon by passing an inflation fluid across the inflation opening.

Embodiment 9 provides the catheter of any one of embodiments 1-8, which further comprises a radiopaque marker at or near the distal end of the catheter shaft.

Embodiment 10 provides the catheter of any one of embodiments 1-9, wherein the catheter is a diagnostic angiography catheter.

Embodiment 11 provides a catheter system comprising:
the catheter of any one of embodiments 1-10; and a guide wire moveable alone a longitudinal direction inside a lumen of the catheter shaft.

Embodiment 12 provides the catheter system of embodiment 11, wherein a diameter of the guide wire ranges from about 0.3 mm to about 1 mm.

Embodiment 13 provides the catheter system of any one of embodiments 11-12, further comprising:
a guide catheter having a guide lumen, wherein the catheter and/or the guide wire is movable alone a longitudinal direction inside the guide lumen.

Embodiment 14 provides the catheter system of embodiment 13, wherein a length of the guide catheter ranges from about 500 mm to about 1100 mm.

Embodiment 15 provides the catheter system of any one of embodiments 13-14, wherein an outer diameter of the guide catheter ranges from about 1.8 mm to about 3 mm.

Embodiment 16 provides the catheter system of any one of embodiments 13-15, wherein the inner diameter of the guide catheter ranges from about 1.6 mm to about 2.4 mm

Embodiment 17 provides a method of navigating the catheter system of any one of embodiments 11-16 from a first blood vessel across a bifurcation point to a second blood vessel, the method comprising:
advancing the guide wire from the first blood vessel toward the second blood until the distal end of the guide wire passes the bifurcation point;
advancing the diagnostic catheter along the guide wire until the inflatable balloon reaches the bifurcation point or the second blood vessel;
inflating the inflatable balloon such that the inflatable balloon anchors the diagnostic catheter to an inner wall of the bifurcation point or the second blood vessel; advancing the guidewire inside the diagnostic catheter further into the second blood vessel;
deflating the balloon to release the diagnostic catheter from the inner wall of the bifurcation point or the second blood vessel; and advancing the diagnostic catheter further into the second blood vessel using the guidewire as a guide.

Embodiment 18 provides the method according to embodiment 17, further comprising:
advancing the diagnostic catheter from the second blood vessel into a third blood vessel, or advancing the diagnostic catheter from the third blood vessel into a fourth blood vessel following the same steps for advancing the diagnostic catheter from the first blood vessel in to the second blood vessel.

Embodiment 19 provides the method of any one of embodiments 17-18, wherein an angle between the first blood vessel and the second blood vessel, an angle between the second blood vessel and the third blood vessel, and/or an angle between the third blood vessel and the fourth blood vessel are about 45 degrees or higher.

Embodiment 20 provides the method of any one of embodiments 17-19, wherein the first blood vessel is the aorta, and the second blood vessel is a common carotid artery, an innominate artery, a subclavian artery, a renal artery, or another peripheral artery connected to the aorta.

Embodiment 21 provides the method of any one of embodiments 18-20, wherein the third blood vessel is an internal carotid artery, external carotid artery, subclavian artery, or vertebral artery.

Embodiment 22 provides the method of any one of embodiments 18-21, wherein the fourth blood vessel is a vertebral artery.

Embodiment 23 provides the method of any one of embodiments 17-22, further comprises advancing a guide catheter over the diagnostic catheter and/or the guide wire.

1. A catheter comprising:
a catheter shaft having at least one curve near a distal end thereof; and
an inflatable balloon coupled to or formed on an exterior surface of a distal region of the catheter shaft.

2. The catheter of claim 1, wherein an outer diameter of the catheter shaft ranges from about 1.2 mm to about 2.5 mm.

3. The catheter of claim 1, wherein an inner diameter of a catheter lumen inside the catheter shaft ranges from about 0.1 mm to about 2 mm.

4. The catheter of claim 1, wherein the at least one of the following applies:
(a) the curve comprises a distal curve, and the inflatable balloon is between the distal curve and a distal end of the catheter shaft,
(b) the curve comprises a distal curve, and the inflatable balloon is between the distal curve and a proximal end of the catheter shaft,
(c) the at least one curve comprises a distal curve and a proximal curve, and the inflatable balloon is between the distal curve and the proximal curve.

5. The catheter of claim 1, wherein a curvature profile of the catheter shaft near the distal end thereof is a curvature profile of a Simmons catheter, a Berenstein catheter, an angle catheter, a tapered angle catheter, a Vitek catheter, a renal double curve catheter, a Mikaelsson catheter, and/or a Cobra catheter.

6. The catheter of claim 1, wherein a distal end of the inflatable balloon is about 100 mm or less from the distal end of the diagnostic catheter, and wherein the distal end of the inflatable balloon is about 2 mm or more from the distal end of the diagnostic catheter.

7. The catheter of claim 1, wherein a length of the balloon along a direction of the catheter shaft ranges from about 5 mm to about 20 mm.

8. The catheter of claim 1, which further comprises an inflation lumen having an inflation opening inside the balloon and an inflation port at a proximal end of the catheter shaft, wherein the inflation lumen and the inflation port inflates or deflates the balloon by passing an inflation fluid across the inflation opening.

9. The catheter of claim 1, which further comprises a radiopaque marker at or near the distal end of the catheter shaft.

10. The catheter of claim 1, wherein the catheter is a diagnostic angiography catheter.

11. A catheter system comprising:
the catheter of claim 1; and
a guide wire moveable alone a longitudinal direction inside a lumen of the catheter shaft.

12. The catheter system of claim 11, wherein a diameter of the guide wire ranges from about 0.3 mm to about 1 mm.

13. The catheter system of claim 11, further comprising:
a guide catheter having a guide lumen, wherein the catheter and/or the guide wire is movable alone a longitudinal direction inside the guide lumen.

14. The catheter system of claim 13, wherein a length of the guide catheter ranges from about 500 mm to about 1100 mm.

15. The catheter system of claim 13, wherein an outer diameter of the guide catheter ranges from about 1.8 mm to about 3 mm.

16. The catheter system of claim 13, wherein the inner diameter of the guide catheter ranges from about 1.6 mm to about 2.4 mm.

17. A method of navigating the catheter system of claim 11 from a first blood vessel across a bifurcation point to a second blood vessel, the method comprising:
advancing the guide wire from the first blood vessel toward the second blood until the distal end of the guide wire passes the bifurcation point;
advancing the diagnostic catheter along the guide wire until the inflatable balloon reaches the bifurcation point or the second blood vessel;
inflating the inflatable balloon such that the inflatable balloon anchors the diagnostic catheter to an inner wall of the bifurcation point or the second blood vessel;
advancing the guidewire inside the diagnostic catheter further into the second blood vessel;
deflating the balloon to release the diagnostic catheter from the inner wall of the bifurcation point or the second blood vessel; and
advancing the diagnostic catheter further into the second blood vessel using the guidewire as a guide.

18. The method according to claim 17, further comprising: advancing the diagnostic catheter from the second blood vessel into a third blood vessel, or advancing the diagnostic catheter from the third blood vessel into a fourth blood vessel following the same steps for advancing the diagnostic catheter from the first blood vessel into the second blood vessel.

19. The method of claim 17, wherein an angle between the first blood vessel and the second blood vessel, an angle between the second blood vessel and the third blood vessel, and/or an angle between the third blood vessel and the fourth blood vessel are about 45 degrees or higher.

20. The method of claim 17, wherein the first blood vessel is the aorta, and the second blood vessel is a common carotid artery, an innominate artery, a subclavian artery, a renal artery, or another peripheral artery connected to the aorta.

21. The method of claim 18, wherein the third blood vessel is an internal carotid artery, external carotid artery, subclavian artery, or vertebral artery.

22. The method of claim 18, wherein the fourth blood vessel is a vertebral artery.

23. The method of claim 17, further comprises advancing a guide catheter over the diagnostic catheter and/or the guide wire.
